(19) United States
(12) Patent Application Publication
(10) Pub. No.: US 2024/0359020 A1
(43) Pub. Date: Oct. 31, 2024

(54) EXTERNAL CUES COMBINED WITH THERAPY TO IMPROVE FUNCTION

(71) Applicant: Boston Scientific Neuromodulation Corporation, Valencia, CA (US)

(72) Inventors: Mahsa Malekmohammadi, Sherman Oaks, CA (US); Lisa Denise Moore, Glendale, CA (US)

(21) Appl. No.: 18/646,302

(22) Filed: Apr. 25, 2024

Related U.S. Application Data
(60) Provisional application No. 63/461,838, filed on Apr. 25, 2023.

(51) Int. Cl. A61N 1/372; A61N 1/05; A61N 1/36

(52) U.S. Cl. CPC A61N 1/37247; A61N 1/0534; A61N 1/36067; A61N 1/36135

(57) ABSTRACT

A system may include a deep brain stimulation (DBS) system and an external user system. The DBS system may include a DBS lead and a DBS neuromodulator configured to use the DBS lead to deliver a DBS therapy to a patient having a neurological disorder. The external user system may include a processor and a user output configured to provide a perceptible output signal to the patient. The processor may be configured to execute a cueing application to implement a cueing routine to cue patient movement. The cueing routine may be configured to control the user output to provide the perceptible output signal.

### PRIORITY CLAIM

This application claims the benefit of U.S. Provisional Application No. 63/461,838 filed on Apr. 25, 2023, which is hereby incorporated by reference in its entirety.

### TECHNICAL FIELD

This document relates generally to medical systems, and more particularly, but not by way of limitation, to systems, devices, and methods for providing therapy and cueing movement for patients with motor disorders.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. By way of example and not limitation, a DBS system may be configured to treat tremor, bradykinesia, and dyskinesia and other motor disorders associated with Parkinson's Disease (PD).

Slowness of movement, called bradykinesia is one of the cardinal symptoms of PD. The PD patient may suffer from general slowness in movement, a reduction in automatic movements or difficulty in initiating or resuming frozen or paused movements. However, a phenomenon termed paradoxical kinesia has been observed, where PD patients may overcome bradykinesia under distinct circumstances. By way of example and not limitation, PD patients may overcome bradykinesia when there is an immediate threat such as an earthquake, an approaching car, a runaway horse, and the like. Cued or timed activity may also be effective in overcoming episodes of freezing or akinesia. For example, a patient who struggles to walk may be able to effortlessly ride a bicycle or smoothly dance to a rhythmic music. A common hypothesis for paradoxical kinesia postulates that the presentation of external sensory triggers is pivotal to elicit significant increase of motor velocity. Distler M, Schlachetzki J C, Kohl Z, Winkler J, Schenk T. Paradoxical kinesia in Parkinson's disease revisited: Anticipation of temporal constraints is critical. Neuropsychologia. 2016 Jun;86:38-44. doi: 10.1016/j.neuropsychologia.2016.04.012. Epub 2016 Apr 15. PMID: 27090103.

Noting that motor movement includes preparing, initiating, and executing the movement, one theory for this paradoxical kinesia is that cognitively-driven and emotionally-driven brain motor pathways facilitate contextually different types of movement, and may use differing pathways through common neuroanatomical structures within the motor system. PD patients therefore may use compensation strategies to address bradykinesia. For example, some PD patients use running to compensate for gait difficulties. Cueing may also be used as a rehabilitation tool. For example, a known device from NexStride™ attempts to compensate for gait difficulties using a device that can be placed on a cane to provide a visual signal (laser light on the ground) and/or a metronome audio signal to cue walking.

A patient who is treated with DBS may have several troublesome symptoms. For example, a PD patient may suffer from several symptoms such as bradykinesia, tremor, rigidity, and freezing of gait (FOG). The focus is often on one or more troublesome symptoms when the DBS therapy is programmed. However, it may not be feasible or practical to resolve all symptoms with one set of programs. For example, a patient may have troublesome tremor and bradykinesia as well as FOG. The programmed DBS may mainly control their tremor while the PD patient's FOG remains poorly controlled. Additional programs may be defined to address other symptoms; however, patients will need to be able to adjust the program based on their needs manually.

There is a need and opportunity to improve and expand DBS efficacy using rehabilitation tools that can improve function in PD using alternate motor pathways.

### SUMMARY

An example (e.g., “Example 1”) of subject matter may include a system comprising a deep brain stimulation (DBS) system and an external user system. The DBS system may include a DBS lead and a DBS neuromodulator configured to use the DBS lead to deliver a DBS therapy to a patient having a neurological disorder. The external user system may include a processor and a user output configured to provide a perceptible output signal to the patient. The processor may be configured to execute a cueing application to implement a cueing routine to cue patient movement. The cueing routine may be configured to control the user output to provide the perceptible output signal.

In Example 2, the subject matter of Example 1 may optionally be configured to further include a controller configured to coordinate the DBS therapy with the cueing routine by: changing to a different DBS program when the cueing routine is implemented; activating a second DBS program, and interleaving the second program with a first DBS program that was active before the second DBS program was activated; changing at least one DBS parameter value when the cueing routine is implemented; or intermittently delivering the DBS therapy to avoid delivering the DBS therapy during the cueing routine.

In Example 3, the subject matter of any one or more of Examples 1-2 may optionally be configured such that the implemented cueing routine relieves a movement disorder or symptom in the patient, and the DBS therapy relieves at least one other symptom of the patient.

In Example 4, the subject matter of any one or more of Examples 1-3 may optionally be configured such that the processor is configured to implement the cueing routine to provide a plurality of perceptible signal instances. The processor may be configured to control timing for providing the plurality of perceptible signal instances.

In Example 5, the subject matter of Example 4 may optionally be configured such that the external user system is configured to automatically adjust an interval between consecutive perceptible signal instances.

In Example 6, the subject matter of any one or more of Examples 1-5 may optionally be configured such that the processor is configured to implement the cueing routine to control a location for the perceptible output signal or a sequence of more than one location for the perceptible signal. The processor may be configured to automatically adjust the location or the sequence of the more than one location.

In Example 7, the subject matter of any one or more of Examples 1-6 may optionally be configured such that the external user system includes a user interface configured to receive a user input for determining when to implement the cueing routine.

In Example 8, the subject matter of any one or more of Examples 1-7 may optionally be configured such that the external user system includes at least one sensor. The cueing application may be configured to use the sensor to determine when to implement the cueing routine.

In Example 9, the subject matter of Example 8 may optionally be configured such that the sensor includes at least one of an accelerometer, a pressure sensor and an exertion sensor. The cueing application may be configured use the sensor to determine at least one of actual patient movement, attempted patient movement or predicted patient movement.

In Example 10, the subject matter of any one or more of Examples 1-9 may optionally be configured such that the patient movement to be cued includes walking.

In Example 11, the subject matter of any one or more of Examples 1-10 may optionally be configured such that the patient movement to be cued includes movement of a hand or arm of the patient.

In Example 12, the subject matter of any one or more of Examples 1-11 may optionally be configured such that the cueing application is configured to determine, infer, or access a patient medication state. The cueing application may be configured to adjust the cueing routine based at least in part on the patient medication state.

In Example 13, the subject matter of any one or more of Examples 1-12 may optionally be configured such that the external system includes a phone, watch, another wearable or an external sensor configured to execute the cueing application to implement the cueing routine. The perceptible output signal used to cue patient movement may include at least one of a sound or a vibration produced by the phone, the watch or the other wearable.

In Example 14, the subject matter of Example 13 may optionally be configured such that the phone, the watch or the other wearable includes an accelerometer. The cueing application may be configured to use the accelerometer to determine when to implement the cueing routine by determining at least one of actual patient movement, attempted patient movement or predicted patient movement. The phone, the watch or the other wearable may be configured to use a location service to implement the cueing routine by determining a location for at least one of actual patient movement, attempted patient movement or predicted patient movement.

In Example 15, the subject matter of any one or more of Examples 1-14 may optionally be configured such that the external system includes at least one of a wearable audio-producing device, a wearable haptic sensation producing device, a wearable visual cue producing device, non-wearable audio-producing speakers, a non-wearable visual cue producing device, one or more wearable sensors or one or more location sensors.

Example 16 includes subject matter (such as a method, means for performing acts, machine readable medium including instructions that when performed by a machine cause the machine to perform acts, or an apparatus to perform). The subject matter may include delivering a deep brain stimulation (DBS) therapy to a patient having a neurological disorder. The DBS therapy may be delivered using a DBS system that includes a DBS neuromodulator and a DBS lead. The subject matter may include cueing patient movement by providing a perceptible output signal to the patient using an external user system that includes a processor and a user interface configured to provide the perceptible output signal. The processor may be configured to execute a cueing application to implement a cueing routine to cue patient movement.

In Example 17, the subject matter of Example 16 may optionally be configured to further include using a controller to coordinate the DBS therapy with the cueing routine by: changing to a different DBS program when the cueing routine is implemented; activating a second DBS program, and interleaving the second program with a first DBS program that was active before the second DBS program was activated; changing at least one DBS parameter value when the cueing routine is implemented; or intermittently delivering the DBS therapy to avoid delivering the DBS therapy during the cueing routine.

In Example 18, the subject matter of any one or more of Examples 16-17 may optionally be configured such that the cueing patient movement relieves a movement disorder or symptom in the patient, and the DBS therapy relieves at least one other symptom of the patient.

In Example 19, the subject matter of any one or more of Examples 16-18 may optionally be configured such that the cueing patient movement includes providing a plurality of perceptible output signal instances. The subject matter may further include controlling timing for providing the plurality of perceptible signal instances.

In Example 20, the subject matter of Example 19 may optionally be configured to further include automatically adjusting an interval between consecutive perceptible signal instances.

In Example 21, the subject matter of any one or more of Examples 16-20 may optionally be configured such that the cueing patient movement includes controlling a location for the perceptible output signal or a sequence of more than one location for the perceptible signal. The location or the sequence of the more than one location may be automatically adjusted.

In Example 22, the subject matter of any one or more of Examples 16-21 may optionally be configured to further include receiving a user input to determine when to implement the cueing routine.

In Example 23, the subject matter of any one or more of Examples 16-22 may optionally be configured to further include using a sensor to determine when to implement the cueing routine.

In Example 24, the subject matter of Example 23 may optionally be configured such that the sensor includes at least one of an accelerometer, a pressure sensor or an exertion sensor. The subject matter may further include using the sensor to determine at least one of actual patient movement, attempted patient movement or predicted patient movement.

In Example 25, the subject matter of any one or more of Examples 16-24 may optionally be configured such that the cueing patient movement includes cueing walking.

In Example 26, the subject matter of any one or more of Examples 16-25 may optionally be configured such that the cueing patient movement includes cueing hand movement or arm movement.

In Example 27, the subject matter of any one or more of Examples 16-26 may optionally be configured to further include determining, inferring or accessing a patient medication state. The cueing routine may be adjusted based at least in part on the patient medication state.

In Example 28, the subject matter of any one or more of Examples 16-27 may optionally be configured such that the external system includes a phone, watch or another wearable configured to execute the cueing application to implement the cueing routine. The perceptible output signal used to cue patient movement may include at least one of a sound or a vibration produced by the phone, the watch or the other wearable.

In Example 29, the subject matter of Example 28 may optionally be configured such that the phone, the watch or the other wearable includes an accelerometer. The cueing application may be configured to use the accelerometer to determine when to implement the cueing routine by determining at least one of actual patient movement, attempted patient movement or predicted patient movement. The phone, the watch or the other wearable may be configured to use a location service to implement the cueing routine by determining a location for at least one of actual patient movement, attempted patient movement or predicted patient movement.

In Example 30, the subject matter of any one or more of Examples 16-29 may optionally be configured such that the external system includes at least one of a wearable audio-producing device, a wearable haptic sensation producing device, a wearable visual cue producing device, non-wearable audio-producing speakers, a non-wearable visual cue producing device, one or more wearable sensors or one or more location sensors.

An example (e.g., “Example 31”) of subject matter may include a non-transitory machine-readable medium including instructions, which when executed by a machine, cause the machine to perform a method comprising: delivering a deep brain stimulation (DBS) therapy to a patient having a neurological disorder. The DBS therapy may be delivered using a DBS system that includes a DBS neuromodulator and a DBS lead. The method may further include cueing patient movement by providing a perceptible output signal to the patient using an external user system that includes a processor and a user input configured to provide the perceptible output signal. The processor may be configured to execute a cueing application to implement a cueing routine to cue patient movement.

In Example 32, the subject matter of Example 31 may optionally be configured such that the method further includes coordinating the DBS therapy with the cueing routine by changing to a different DBS program when the cueing routine is implemented, activating a second DBS program, and interleaving the second program with a first DBS program that was active before the second DBS program was activated, changing at least one DBS parameter value when the cueing routine is implemented, or intermittently delivering the DBS therapy to avoid delivering the DBS therapy during the cueing routine.

In Example 33, the subject matter of any one or more of Examples 16-32 may optionally be configured such that the cueing patient movement includes providing a plurality of perceptible output signal instances. The method may further include controlling timing for providing the plurality of perceptible signal instances.

In Example 34, the subject matter of any one or more of Examples 16-33 may optionally be configured such that the cueing patient movement includes controlling a location for the perceptible output signal or a sequence of more than one location for the perceptible signal. The location or the sequence of the more than one location may be automatically adjusted.

In Example 35, the subject matter of any one or more of Examples 16-34 may optionally be configured such that the method further comprises determining, inferring or accessing a patient medication state, and adjusting the cueing routine based at least in part on the patient medication state.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a system configured to deliver DBS therapy and to cue patient movement.

FIG. 5 illustrates, by way of example and not limitation, a system configured to deliver DBS therapy and to cue patient movement.

FIG. 6 illustrates, by way of example and not limitation, an external system used to cue system movement that may be used for the external system in FIG. 4.

FIG. 7 illustrates, by way of example and not limitation, a method for delivering a cueing routine with a DBS therapy.

FIG. 8 illustrates, by way of example and not limitation, a method for calibration the cueing system.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments disclosed herein improve function in patients with motor disorders using systems, devices, and methods for providing therapy (e.g., DBS) and cueing movement. Various embodiments introduce external cues and strategically combine them with DBS therapy. The external cues may be introduced from external sensors and other components. For example, the cueing may be used with DBS stimulation to relieve symptoms that may remain inadequately controlled by the DBS therapy acting alone. The DBS therapy may be changed to accommodate times with cueing in view of times without cueing. For example, a DBS program may be switched to activate a different neural pathway/circuit/neural population when cueing is provided. In an example, recalibration may be used to improve a cueing therapy and/or a DBS therapy that may have lost its effectiveness over time (e.g., due to habituation).

Cued movement may include a stimulus presented with spatial and/or temporal component(s) for use in initiating and/or executing movement. Compared to self-initiated movement, cued movement may improve the time and peak velocity of the movement. Spatial cue components provide “location” information, such as lines on the floor that identify targets (e.g., “place your foot here”) for the patient's steps. Temporal cue components provide “timing” information, such as a rhythmic metronome-like sound or other signal that triggers “move now”. Cues may include both spatial and temporal components such as, by way of example and not limitation, a line on the floor that may appear (e.g., flash) or become more vivid to provide the “move now” cue with the “place your foot here” cue. The spatial cue components may, but do not have to be, evenly spaced and the temporal components may, but do not have to be, evenly spaced. For example, a steady state walking gait may include evenly spaced spatial and temporal components. However, a walking routine may ramp up to speed and ramp down to speed such that the cues may include progressively increasing or decreasing spatial components and/or progressively increasing or decreasing temporal components. Other movements, such as eating or phone use by way of example and not limitation, may be cued, and the cueing routines may be adjusted to accommodate the needs of the patient.

Cues may be provided using senses such as touch, sound or sight. Somatosensory stimuli (e.g., perceived by the skin) may be used. For example, a patient may enable “walk assist” as part of their system to overcome freezing of gait (FOG). A DBS patient may use a DBS system that includes a digital phone application, an external wearable, and a DBS implant. Accelerometers in the DBS implant, external wearable, and/or phone can be used to detect that patient is predicted to be walking and has frozen. Additionally, or alternatively, other external sensors such as, but not limited to pressure sensors in the shoes or position sensors in the socks, can be used to determine a posture of the patient and/or whether the patient is walking or predicted to be walking. When patient is predicted to be walking, and/or when a freeze or hesitation or shuffling is detected, the mobile phone and/or an external wearable can vibrate at a speed consistent with the patient's normal walking speed to help prevent/overcome freezing episode. The speed of vibration can be auto calibrated to the user based on walking speed derived from predicted heel strikes that may be detected by external wearables (especially foot or leg worn systems). The speed of vibration may be adjusted by the patient to be slower or faster based on the patient's needs. Additionally, or alternatively, an auditory cue may be sent from the phone, external wearable, or to an attached ear bud, or bone conduction hearing tools. Visual cues may be provided. For example, a flashlight on the phone or phone screen may strobe to cue movement or on digital eyeglasses worn by the patient. The system may assist with user interactions with the phone by changing (e.g., strobing) the brightness or size of the touch screen elements to be touched by the patient.

Patient therapies may be modulated based on predicted outcomes of patient treatment state. Freezing episodes may be tracked or anticipated based on the movement patterns preceding the freezing episode. The tracked freezing episodes in combination with patient treatment (e.g., the patient's medication, stimulation, and walk assist therapy) may be used to predict how to modulate therapy based on patient medication state. For example, when a patient is in a meds high state they may need less or no “walk assist” compared to a meds low state. A similar situation may occur when a patient is using one program versus another. A schedule (e.g., a time of day or night) of the patient may be a component of the decision. For example, the patient is likely to engage in eating behavior (i.e., a repetitive motion likely to induce bradykinesia) around meal time.

A cueing therapy may be implemented when a bradykinesia episode is detected or may be implemented based on a detected patient state where it is detected that the current patient state (e.g., treatment state) leads to a higher likelihood of bradykinesia (e.g., FOG episode). For example, the cueing system may be triggered when the patient stands upright for a given treatment state, and may not be triggered when the patient stands for another treatment state.

Medication state is a nonexclusive example of a treatment state on which the cueing therapy may be based. It is noted that a drug concentration varies, as it generally increases as a dose is absorbed and then decreases with drug clearance through metabolism and elimination. Further, a drug concentration may fluctuate over the course of multiple doses of the medicine. For example, drug concentration may take multiple doses (e.g., 5 doses) before the drug concentrations reach a stead state of fluctuations.

Various embodiments of the present subject matter account for medication state when implementing a cueing therapy. The medicated state may be a “medicated” or “unmedicated” state, or may be more than just a medicated/unmedicated states as a concentration of a single medication fluctuates based on the dose, the dose interval, the absorption rate, and the clearance rate (e.g., metabolism, elimination). Furthermore, drug metabolism rate varies for different drugs, and the metabolism rate for the same drug may vary among patients. Factors that may affect drug metabolism rate include genetics, coexisting disorders, diet, and drug interactions. Many patients may be taking multiple drugs, with different drugs being taken at different times and different doses. The medication state may be acquired for each drug individually, or may be acquired for the drugs in combination.

Various embodiments are capable of predicting and/or incorporating the patient's medication state. The medication state may be determined using sensor(s), timer(s), patient or other user input, and various combinations thereof. By way of example and not limitation, examples of sensor(s) that may infer a medication state may include neural sensors configured for use in detecting a characteristic change in sensed neural activity. The neural sensors may be used in detecting change(s) in evoked neural activity. Other sensor examples include chemical sensors for detecting molecules withing the body (e.g., dopamine sensors). Examples of sensor(s) may include external and/or internal sensor(s) configured for use in detecting a characteristic shift in the treated condition (e.g., accelerometer(s) capable of detecting movements for use in detecting a characteristic shift in movement disorders such as tremor/bradykinesia/dyskinesia), or blood pressure, pulse rate or electrocardiogram (ECG) sensors that may be used to monitor cardiovascular health, or sensor(s) that may be used to infer changes in pain (e.g., galvanic skin response, heart rate, blood pressure, posture, motions and/or facial recognition sensor(s)). Sensor(s) may be configured to detect the concentration of the medicine within the patient. Some embodiments may infer a medication state based on changes in the patient's routine for interacting with their phone or other device. This inference may be combined with a timer or clock, a patient's medication schedule, an activity or posture sensor, a location sensor, and/or other sensors or inputs.

The system may associate programmable timer(s) with the patient's medication schedule to determine a medication state. A system may essentially assume that the patient is taking medication as directed, and is expected to have typical concentration variations in view of the scheduled dosing for the medication. The typical concentration variations may be determined based on evaluations of variations in the patient and/or based on evaluations of patient populations. The timers may be incorporated into a clinical programmer, a remote control, a patient's personal device (e.g., smart phone or tablet), or the medical device (e.g., implantable device).

The medication state may be determined using patient input, which may include a notification by the patient through interaction with the medical device, the remote control, the patient's personal device, and the like to indicate that medication was taken. The patient input may indicate the dose and time that the medication was taken. The input may be provided by another user such as a clinician, a family member, or other healthcare givers. Mood assessments and cognitive scores may infer medication state.

Cueing may be used to improve function in patients with FOG. Bradykinesia other than FOG may be cued. For example, bradykinesia affecting hand activity may be detected from a wrist worn wearable or by a user input from a clinician, caregiver, patient or other user. The system may detect that the patient is moving one arm and is continuing to do so regularly over a set period (two minutes) to eat. The system may be configured to automatically deliver cues through vibrations on a wrist worn wearable in an effort to reduce patient's bradykinesia.

Various embodiments disclosed herein include DBS and cueing systems. The DBS and cueing systems may be integrated together. For example, a DBS therapy may be changed when there is an active cueing routine. By way of example and not limitations, an accelerometer in an implanted DBS neuromodulator may be used to control the cueing system and/or DBS therapies may be modified based on whether patient movement is being cued. For example, the system may change to a different DBS program when a cueing routine is implemented. The system may activate another (e.g., second) DBS program, and the second program may be interleaved with a first DBS program that was active before the second program was activated. The system may change at least one DBS parameter value when the cueing routine is implemented. The system may intermittently deliver the DBS therapy to avoid delivering the DBS routine during the cueing routine. By way of example and not limitation, the DBS routine may be turned off and just cueing may be used to provide relief for the newly emerged troublesome symptoms. And once the need is no longer present, the cueing system may be turned off ad the DBS routine may be turned back on. More complex examples may set or schedule times for different combinations of DBS and cueing states. By way of example and not limitation, where the DBS states are ON and OFF and the CUE STATES are ON and OFF, the system may include an alternative schedule of DBS ON/CUE OFF, DBS OFF/CUE ON, DBS ON/CUE OFF until the need is no longer present. The schedule may include times where both DBS and CUE are ON, and may include times where both DBS and CUE are OFF. More complex examples may be implemented using more than two states (ON/OFF).

A cueing routine may be changed, started or stopped in the presence of an active DBS therapy. For example, a patient may have a DBS treatment that assists with their movement but impairs speech. During normal therapy, this patient may not need cueing routines. However, if the patient has switched to a DBS therapy more conducive to speech, the cueing routine may be used to assist them with their movement. In another example, the patient may have difficulties with a certain activity or movement at a set time of day that is not notably connected to medication. During this time of day, the cueing routine can activate when the activity is detected. The system may determine that the DBS system has exhausted its ability to improve the patient and that the cueing routine is being used to compensate.

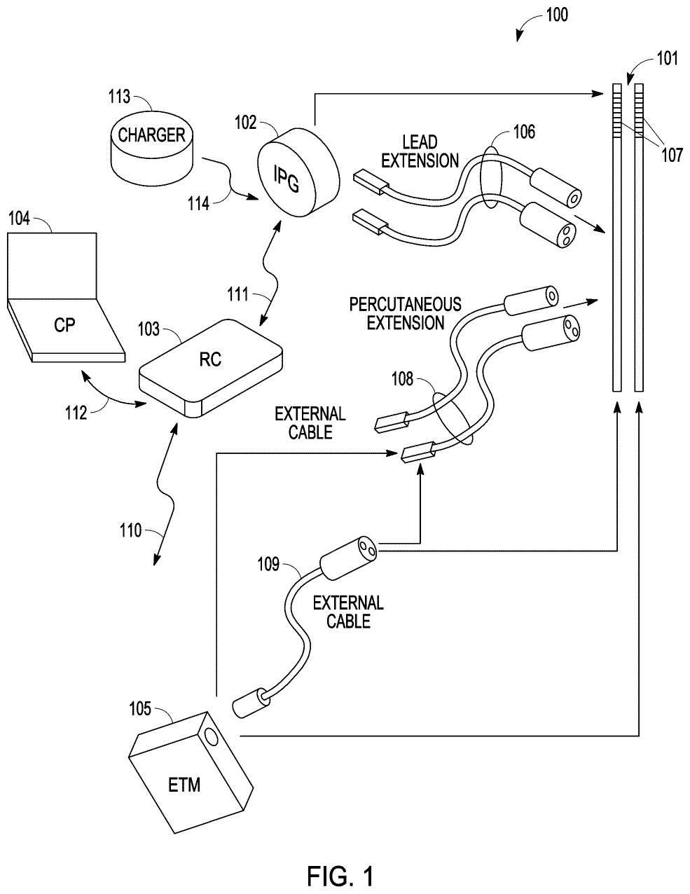

FIG. 1 illustrates, by way of example and not limitation, an electrical stimulation system 100, which may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 107. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode). Other types of leads may be used. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 108 and external cable 109 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 110. The RC 103 may be used to telemetrically control the IPG 102 via a bi-directional RF communications link 111. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 112 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 113 may be a portable device used to transcutaneous charge the IPG 102 via a wireless link such as an inductive link 114. Once the IPG 102 has been programmed, and its power source has been charged by the external charger or otherwise replenished, the IPG 102 may function as programmed without the RC 103 or CP 104 being present.

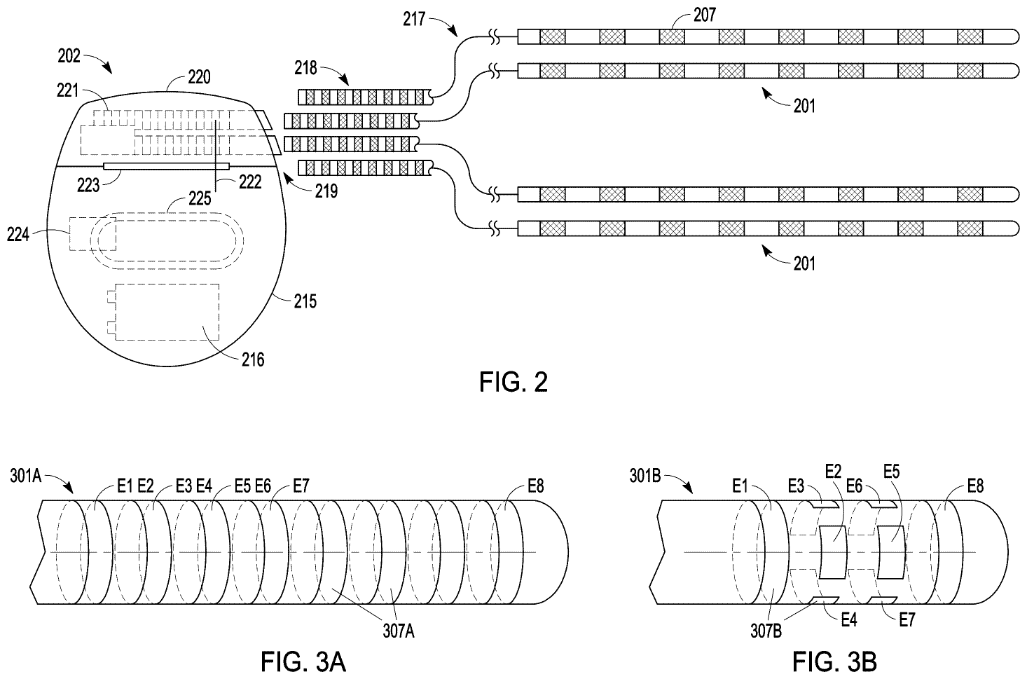

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 215 that holds the circuitry and a battery 216 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 207 for delivering electrostimulation energy, recording electrical signals, or both. In some examples, the leads 201 can be rotatable so that the electrodes 207 can be aligned with the target neurons after the neurons have been located such as based on the recorded signals. The electrodes 207 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in PD for example, the IPG 202 is typically implanted under the patient's clavicle (collarbone). The leads 201 (which may be extended by lead extensions, not shown) can be tunneled through and under the neck and the scalp, with the electrodes 207 implanted through holes drilled in the skull and positioned for example in the subthalamic nucleus (STN) and the pedunculopontine nucleus (PPN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 207) and a case electrode. A bipolar stimulation current can be delivered between two lead-based electrodes (e.g., two of the electrodes 207). Stimulation parameters typically include current amplitude (or voltage amplitude), frequency, pulse width of the pulses or of its individual phases, electrodes selected to provide the stimulation, polarity of such selected electrodes (i.e., whether they act as anodes that source current to the tissue), or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 307A disposed at least partially about a circumference of the lead 301A. The electrodes 307A may be located along a distal end portion of the lead. As illustrated herein, the electrodes 307A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 307B including ring electrodes such as E1 at a proximal end and E8 at the distal end. Additionally, the lead 301 also may include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be used on a lead with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length of the lead 219.

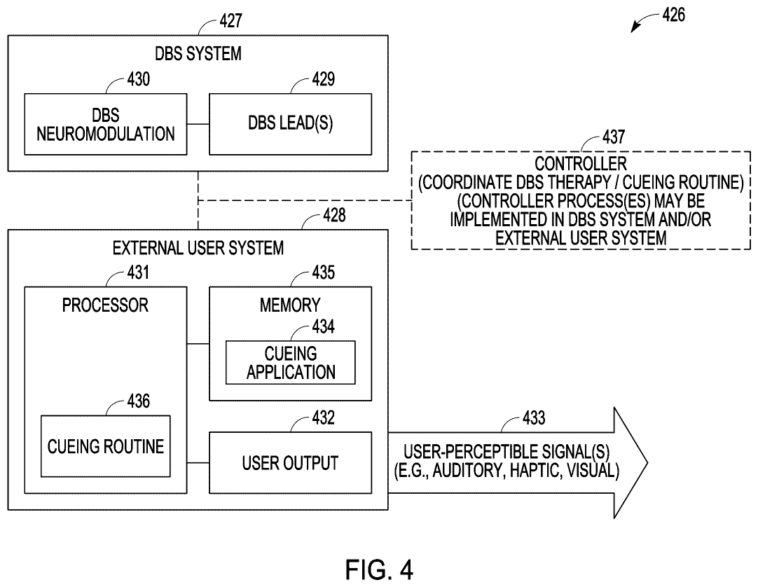

FIG. 4 illustrates, by way of example and not limitation, a system configured to deliver DBS therapy and to cue patient movement. The illustrated system 426 includes a DBS system 427, such as but not limited to systems similar to the systems illustrated in FIGS. 1 through 3B, and an external user system 428. The external user system 428 may be a single device such as, but not limited to, a patient remote-control device, a phone, or a wearable device such as an ear bud, a headset, a hearing assistance device such as, but not limited to, a hearing aid, a watch, a finger worn ring, a wrist or an armband. The external user system may include more than one device such as, but not limited to, a phone and one or more separate auditory devices like ear buds, headsets, hearing aids, speakers, separate somatosensory device(s) such as haptic devices (vibration devices other tactile and/or kinesthetic producing device), or separate sensor(s) like accelerometers, motion sensors, postures sensors, heart rate sensors, blood pressure sensors, respiratory sensors, neural activity sensors, electromyography (EMG) sensors or galvanic skin response (GSR) sensors. The DBS system 427 may include at least one DBS lead 429 and a DBS neuromodulator 430 configured to use the DBS lead(s) 429 to deliver a DBS therapy to a patient having a neurological disorder. The external user system 428 may include a processor 431 and a user output 432 configured to provide at least one perceptible output signal 433 to the patient. The processor 431 may be configured to execute a cueing application 434, which may be stored in a memory 435, to implement a cueing routine 436 to cue patient movement. The cueing routine may be configured to control the user output 432 to provide the perceptible output signal(s) 433. The perceptible output signal may include vibration, sound, light such as a changing light on the phone screen, projected light (e.g., laser light) such as may be used to provide target lines in front of the patient, augmented reality or virtual reality. The implemented cueing routine relieves a movement disorder in the patient, and the DBS therapy relieves at least one other symptom of the patient.

The system 426 illustrated in FIG. 4 may include a controller 437 configured to implement controller processes. The controller processes may be implemented in one device separate from either the DBS system 427 or the external user system 428. The controller processes may be implemented in one of the DBS system 427 or external user system 428. The controller processes may be distributed among multiple devices including at least some device(s) in the DBS system 427 and/or external system 428.

The controller processes may be configured to determine when to implement the cueing routine and/or a specific cueing routine. For example, the external user system 428 may include a user interface configured to receive a user input for determining when to implement the cueing routine. The external user system 428 may include at least one sensor and the cueing application may be configured to use the sensor to determine when to implement the cueing routine. For example, the sensor(s) may include at least one of an accelerometer(s), a pressure sensor(s), or an exertion sensor(s) used by cueing application to determine at least one of actual patient movement, attempted patient movement or predicted patient movement. By way of non-limiting illustration, pressure sensors may be within, on or integrated with the patient's socks or insoles and used to sense when the patient is attempting to walk. For example, the system may be configured to anticipate that the patient will walk and trigger the cueing routine if the patient stands or sits up. The system may be configured to detect attempts to move and/or detect tremor indicative that movement is difficult or slow. The system may be configured to detect actual movement. If actual movement is good, the system may determine not to deliver the cueing stimulus/stimuli, or may change the cueing stimulus/stimuli to match the actual movement.

The system may be configured to cue walking such as may be useful to alleviate or overcome festination, hesitance and/or freezing of gait (FOG). The system may be configured to cue hand or arm movement of the patient, such as may be useful to assist with eating or using a phone or other mobile device. For example, the system may detect that the screen of the phone or other mobile device is unlocked or that the device is not sleeping, and automatically implement the cueing routine to assist with the arm or hand movement. Other motions such as sitting or standing may be cued.

The external user system 428 may include a phone or a watch (or other wearable) configured to execute the cueing application to implement the cueing routine. The perceptible output signal 433 used to cue patient movement may include at least one of a sound or a vibration produced by the phone or the watch. The phone or the watch may include an accelerometer and the cueing application may be configured to use the accelerometer to determine when to implement the cueing routine by determining at least one of actual patient movement, attempted patient movement or predicted patient movement. The phone or the watch may be configured to use a location service (e.g., a GPS, Wi-Fi, or cell tower determined location) to implement the cueing routine by determining at least one of actual patient movement, attempted patient movement or predicted patient movement. The external system may include at least one of a wearable audio-producing device such as ear buds, headphones, or hearing aid, a wearable haptic sensation producing device, non-wearable audio-producing speakers, one or more wearable sensors or one or more location sensor.

The controller processes may be configured to coordinate the DBS therapy and the cueing routine. By way of example and not limitation, the controller may coordinate the DBS therapy and the cueing routine by changing to a different DBS program when the cueing routine is implemented, changing at least one DBS parameter value when the cueing routine is implemented, or intermittently delivering the DBS therapy to avoid delivering the DBS therapy during the cueing routine.

The processor 431 may be configured to control timing for providing a plurality of instance of the perceptible signal. For example, the audio, visual and/or haptic signal may be provided with cadence or a steady rhythm such as a metronome. The interval between successive signals may change, such as to become progressively faster, progressively slower, or to cycle between or among two or more different intervals between successive signals. For example, the external user system may be configured to automatically adjust an interval between consecutive perceptible signal instances and/or may be configured to respond to user input to adjust the interval between consecutive perceptible signal instances.

The processor 431 may be configured to control the location (spatial component) for delivering the perceptible signal. For example, the processor may be configured to implement the cueing routine to determine a location for the perceptible output signal or a sequence of more than one location for the perceptible signal. For example, left-side and right-side signals (left/right vibrations, left/right auditory sounds, and/or left/right light such as projected light) may be used to encourage left and right steps, accordingly.

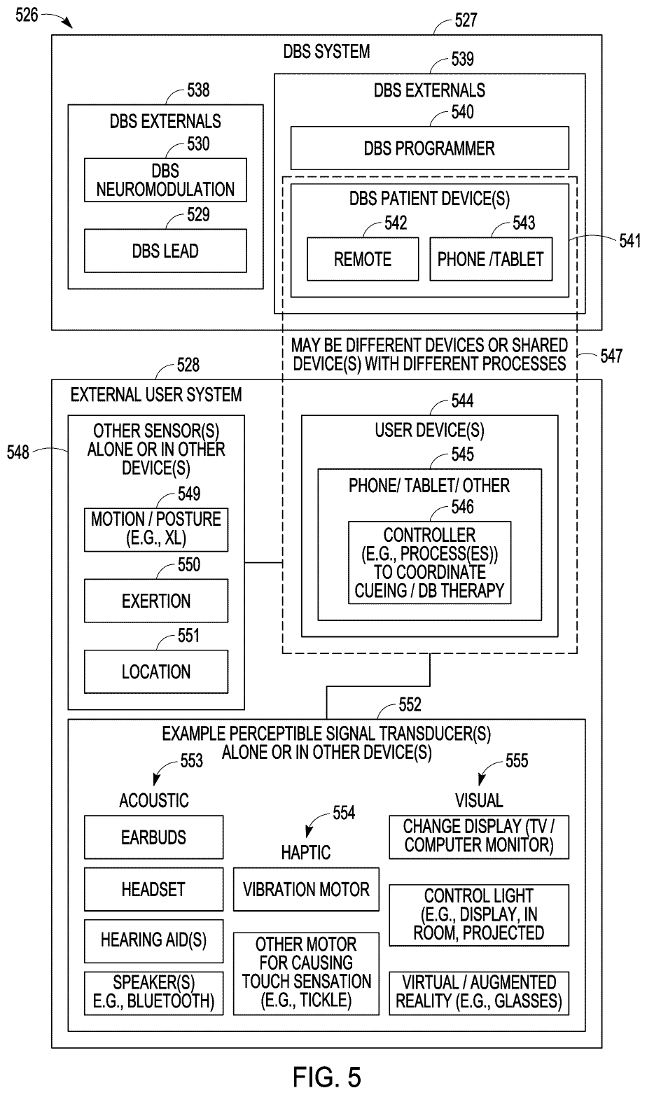

FIG. 5 illustrates, by way of example and not limitation, a system configured to deliver DBS therapy and to cue patient movement. The illustration of the system 526 provides additional detail, as a nonlimiting example, compared to the system 426 illustrated in FIG. 4. The system 526 may include a DBS system 527 and an external user system 528.

The illustrated DBS system 527 includes DBS implant(s) 538 and DBS external(s) 539. The DBS implant(s) 538 may include a DBS neuromodulator 530 such as IPG 102 in FIG. 1 and a DBS lead(s) 529 such as leads 101, 201, 301A or 301B in FIGS. 1, 2, 3A and 3B. The DBS implant(s) 538 may include sensor(s) (not shown) such as posture or activity sensors using accelerometer sensor(s), acoustic sensor(s) or impedance sensor(s). The DBS implant(s) 538 may also include other sensors, such as electrical sensors configured to sense neural activity (such as but not limited to evoked compound action potential (ECAPs), Evoked Resonant Neural Activity (ERNA) or other potentials), and/or muscle activity. The DBS external(s) 539 may include a DBS programmer 540 such as CP 104 in FIG. 1. The DBS external(s) 539 may include DBS patient device(s) 541 such as a patient remote control 542 (e.g., RC 103 in FIG. 1) and/or a phone or tablet 543.

The illustrated external user system 528 may include user devices 544 such as a phone, tablet or other device 545. The illustrated user device(s) 544 may include a controller 546, providing controller processes to coordinate the cueing routine and DBS therapy. As indicated by the dotted line 547, the DBS patient device(s) 541 in the DBS system 527 and the user device(s) 544 in the external user system 528 may include a same device to provide DBS processes and controller processes, or may include different devices such that a device configured provide DBS functions does not also provide the controller processes. Where different devices are used, the user device(s) operating to provide controller processes may communicate with the DBS patient device(s) to control or otherwise coordinate the cueing routine and the DBS therapy.

The illustrated external user system 528 may include other sensor(s) 548 that may be included in device(s) or as standalone sensor(s). Sensor examples include motion or posture sensor(s) 549 such as may include an accelerometer. Sensor examples include exertion sensor(s) 550 such as heart rate sensors, blood pressure sensors, respiration sensors, nerve activity sensors, muscles sensors (EMG), chemical sensors, galvanic skin response (GSR) sensors, and acoustic sensors that may detect grunting or breathing indicative of expended effort. Sensor examples include location sensor(s) 551 which may include locations services such as GPS, Wi-Fi or cellular tower, or may include beacons indicating proximity to a location such as a chair or a particular room (e.g., bathroom, kitchen, bedroom) in a house.

The illustrated external user system 528 may include perceptible signal transducer(s) 552, which may be integrated with other device(s) or may be standalone transducer(s). The signal transducer(s) 552 may include acoustic transducer(s) 553, haptic transducer(s) 554 or visual transducer(s) 555. Acoustic transducer examples 553 include earbuds, headset, hearing device(s) such as hearing aid(s), and/or speaker(s) such as Bluetooth speakers. Haptic transducer examples 554 include a vibration motor, or another motor for causing a touch sensation such as tickle. Visual transducer examples 555 may include a change to a display such as a television or computer monitor, a controlled light such as a light on a display or room light, or projected light, or a virtual augmented reality that may be provided using glasses.

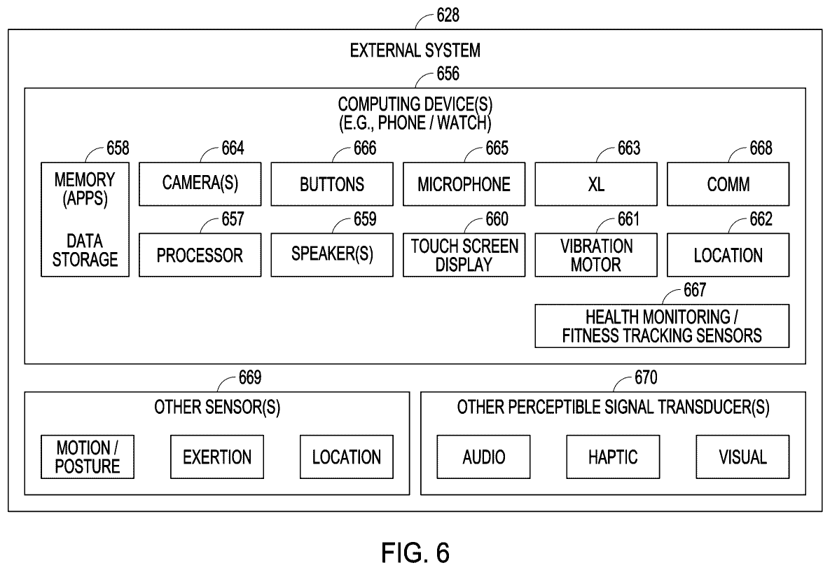

FIG. 6 illustrates, by way of example and not limitation, an external system used to cue system movement that may be used for the external system in FIG. 4. The external system 628 may include one or more computing devices 656. The computing device(s) 656 may include a phone, a tablet, and/or a wearable such as a watch, by way of example. The computing device(s) may include a number of features that may be used by the system to cue system movement. For example, the computing device(s) 656 (e.g., phone, watch, and the like) may include a processor 657 and a memory 658 including apps to be implemented by the processor to perform various processes. The memory 658 may provide data storage for the external system. Other features of the computing device(s) 656 may include at least one of speaker(s) 659 to produce acoustic signals, a touch screen display 660 for providing a user interface used to receive user inputs and provide visual outputs, and a vibration motor 661 that may be used to provide a haptic output. The computing device(s) 656 may include feature(s) capable of being used alone or in various combinations to detect a location or motion 662, which may be used to determine at least one of actual patient movement, attempted patient movement or predicted patient movement. For example, accelerometer(s) (XL) 663, gyroscope, camera(s) 664, microphone(s) 665 and/or location service(s) 662 may be used, with or without a clock and information about the patient's normal activities at different time(s) and/or location(s), to determine at least one of actual patient movement, attempted patient movement or predicted patient movement. The location service(s) 662 may use global positioning system (GPS), Wi-Fi and cellular towers (e.g., triangulation of Wi-Fi access points and/or cellular towers), Bluetooth beacons or Radio Frequency Identification (RFID). A determined location may be used to trigger or adjust cueing. For example, XLs 663 can detection motion and/or posture. Microphones 665 can be used to detect patient effort (breathing, grunts) and sound at the patient's location that may be used to identify the patient's location. Cameras 664 may be used to detect motion, location, and patient effort, such as via a shaky image, facial feature(s) of the patient, visual detection of a bradykinesia event. The touch screen display 660 may be used to determine attempted use of the computing device (e.g., phone) via an unlocked screen or other interaction with the device via the touch screen display or other button(s) 666 on the device. The computing device(s) 656 may include health monitoring/fitness tracking sensor(s) 667 that may make use of other sensor(s) in the device. Health monitoring/fitness tracking sensor(s) 667 may be configured to detect at least an estimate of heart rate, ECG, blood pressure, oxygen, steps, sleep, exercise, stress, and the like. A more exhaustive list of sensor(s) of computing device(s) may include any one or various combinations of an accelerometer, a gyroscope, a magnetometer/compass, a barometric pressure sensor, a body temperature sensor, a heart rate monitor, an oximetry sensor, an ambient light sensor, a bioimpedance sensor, a proximity sensor, an orientation sensor, a pedometer, a calorie counter, an ECG sensor, a gesture sensor, a UV sensor, an electrodermal activity sensor, a skin conductance sensor, and a GPS sensor.

The computing device(s) 656 may include communication technology 668 (e.g., Wi-Fi, Bluetooth) for use to communicate with other computing device(s), other sensor(s), and/or other perceptible signal transducer(s). Other sensor(s) 669 may include other motion and/or posture sensors, other exertion sensor(s), and other sensor(s) for detecting location (e.g., beacon, such as within range of a Bluetooth device). Other perceptible signal transducer(s) 670 may include audio device (e.g., speakers, headsets, earbuds, hearing aids), haptic device(s) (e.g., vibration motor or other devices for provide a tactile and/or kinesthetic sensation), and/or visual device(s) (e.g., lasers, computer or television monitor, projection system, augmented reality or virtual reality).

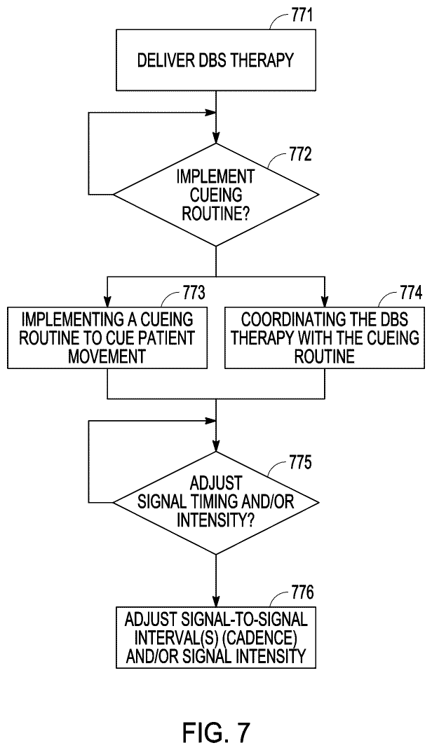

FIG. 7 illustrates, by way of example and not limitation, a method for delivering a cueing routine with a DBS therapy. At 771, a DBS therapy may be delivered. For example, the DBS therapy may be configured to relieve a movement disorder in the patient. At 772, it is determined whether to implement a cueing routine. This determination may be based on a user input and/or sensed data. Examples of sensed data include sensors configured to determine actual patient movement, attempted patient movement or predicted patient movement. If it is determined that a cueing routine should be implemented, the process may continue to 773 to implement the curing routine to cue patient movement.

An external user system may include a processor configured to execute a cueing application to implement a cueing routine to cue patient movement using a perceptible output signal. The cueing patient movement routine may include determining a location for the perceptible output signal or a sequence of more than one location for the perceptible signal, and timing for delivering the perceptible output signal.

In some embodiments as illustrated at 774, the DBS therapy may be coordinated with the cueing routine. The method may further determine, at 775, whether to adjust signal timing, location and/or intensity of the perceptive signals used in the implemented cueing routine. The signal-to-signal interval(s) (e.g., cadence of perceptible signals) and/or signal intensity may be adjusted at 776.

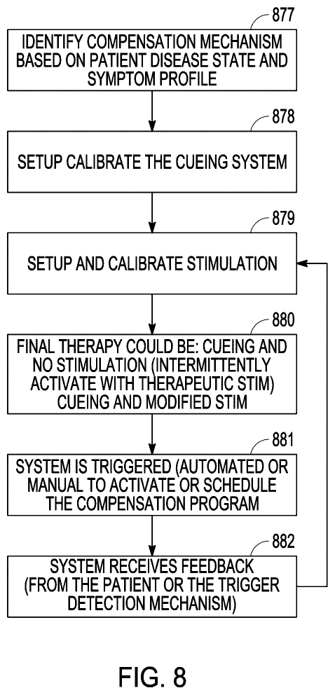

FIG. 8 illustrates, by way of example and not limitation, a method for calibration the cueing system. At 877, the user may identify one or more potential compensation mechanisms based on the patient disease state and symptom profile. The user then may set up calibrate the cueing system 878 (e.g., when and where to deliver the perceptible signal). At 879, the user may setup and calibrate the stimulation (e.g., DBS stimulation). The final therapy may be cueing without stimulation (e.g., via an intermittent use of cueing during times without therapy), or cueing with a modified stimulation 880. The system may be triggered to activate or schedule the compensation program 881. The triggering of the compensation program (e.g., cueing routine) may be automated or manual. At 882, the system may receive feedback from the patient or the rigger detection mechanism, which may be used to modify the setup and the calibration of the stimulation.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
delivering a deep brain stimulation (DBS) therapy to a patient having a neurological disorder, wherein the DBS therapy is delivered using a DBS system that includes a DBS neuromodulator and a DBS lead; and
cueing patient movement by providing a perceptible output signal to the patient using an external user system that includes a processor and a user interface configured to provide the perceptible output signal, wherein the processor is configured to execute a cueing application to implement a cueing routine to cue patient movement.

2. The method of claim 1, further comprising using a controller to coordinate the DBS therapy with the cueing routine by:
changing to a different DBS program when the cueing routine is implemented;
activating a second DBS program, and interleaving the second program with a first DBS program that was active before the second DBS program was activated;
changing at least one DBS parameter value when the cueing routine is implemented; or
intermittently delivering the DBS therapy to avoid delivering the DBS therapy during the cueing routine.

3. The method of claim 1, wherein the cueing patient movement relieves a movement disorder or symptom in the patient, and the DBS therapy relieves at least one other symptom of the patient.

4. The method of claim 1, wherein the cueing patient movement includes providing a plurality of perceptible output signal instances, the method further including controlling timing for providing the plurality of perceptible signal instances.

5. The method of claim 4, further comprising automatically adjusting an interval between consecutive perceptible signal instances.

6. The method of claim 1, wherein the cueing patient movement includes controlling a location for the perceptible output signal or a sequence of more than one location for the perceptible signal and automatically adjusting the location or the sequence of the more than one location.

7. The method of claim 1, further comprising receiving a user input to determine when to implement the cueing routine.

8. The method of claim 1, further comprising using a sensor to determine when to implement the cueing routine.

9. The method of claim 8, wherein the sensor includes at least one of an accelerometer, a pressure sensor or an exertion sensor, the method further including using the sensor to determine at least one of actual patient movement, attempted patient movement or predicted patient movement.

10. The method of claim 1, wherein the cueing patient movement includes cueing walking.

11. The method of claim 1, wherein the cueing patient movement includes cueing hand movement or arm movement.

12. The method of claim 1, further comprising determining, inferring or accessing a patient medication state, and adjusting the cueing routine based at least in part on the patient medication state.

13. The method of claim 1, the external system includes a phone, watch or another wearable configured to execute the cueing application to implement the cueing routine, and the perceptible output signal used to cue patient movement includes at least one of a sound or a vibration produced by the phone, the watch or the other wearable.

14. The method of claim 13, wherein:
the phone, the watch or the other wearable includes an accelerometer and the cueing application is configured to use the accelerometer to determine when to implement the cueing routine by determining at least one of actual patient movement, attempted patient movement or predicted patient movement, or
the phone, the watch or the other wearable is configured to use a location service to implement the cueing routine by determining a location for at least one of actual patient movement, attempted patient movement or predicted patient movement.

15. The method of claim 1, wherein the external system includes at least one of a wearable audio-producing device, a wearable haptic sensation producing device, a wearable visual cue producing device, non-wearable audio-producing speakers, a non-wearable visual cue producing device, one or more wearable sensors or one or more location sensors.

16. A non-transitory machine-readable medium including instructions, which when executed by a machine, cause the machine to perform a method comprising:
delivering a deep brain stimulation (DBS) therapy to a patient having a neurological disorder, wherein the DBS therapy is delivered using a DBS system that includes a DBS neuromodulator and a DBS lead; and
cueing patient movement by providing a perceptible output signal to the patient using an external user system that includes a processor and a user input configured to provide the perceptible output signal, wherein the processor is configured to execute a cueing application to implement a cueing routine to cue patient movement.

17. A system, comprising:
a deep brain stimulation (DBS) system including a DBS lead and a DBS neuromodulator configured to use the DBS lead to deliver a DBS therapy to a patient having a neurological disorder; and
an external user system including a processor and a user output configured to provide a perceptible output signal to the patient, wherein the processor is configured to execute a cueing application to implement a cueing routine to cue patient movement, wherein the cueing routine is configured to control the user output to provide the perceptible output signal.

18. The system of claim 17, further comprising a controller configured to coordinate the DBS therapy with the cueing routine by:
changing to a different DBS program when the cueing routine is implemented;
activating a second DBS program, and interleaving the second program with a first DBS program that was active before the second DBS program was activated;
changing at least one DBS parameter value when the cueing routine is implemented; or
intermittently delivering the DBS therapy to avoid delivering the DBS therapy during the cueing routine.

19. The system of claim 17, wherein the implemented cueing routine relieves a movement disorder or symptom in the patient, and the DBS therapy relieves at least one other symptom of the patient.

20. The system of claim 17, wherein the processor is configured to implement the cueing routine to provide a plurality of perceptible signal instances, and control timing for providing the plurality of perceptible signal instances.
